(19) United States
(12) Patent Application Publication
(10) Pub. No.: US 2017/0014516 A1
(43) Pub. Date: Jan. 19, 2017

(54) COMPOSITIONS AND METHODS FOR BIRDS

(71) Applicant: DUPONT NUTRITION BIOSCIENCES APS, Copenhagen (DK)

(72) Inventors: Lars W. Petersen, Muskego, WI (US); Gregory R. Siragusa, Waukesha, WI (US); Firmin G. Delago, Milwaukee, WI (US); Dennis C. Gainey, Leesville, SC (US)

(73) Assignee: DUPONT NUTRITION BIOSCIENCES APS, Copenhagen (DK)

(21) Appl. No.: 14/853,385

(22) Filed: Sep. 14, 2015

Related U.S. Application Data
(63) Continuation of application No. 13/446,720, filed on Apr. 13, 2012, now abandoned.
(60) Provisional application No. 61/475,540, filed on Apr. 14, 2011.

(51) Int. Cl. A61K 47/36; A61K 47/26; A61K 35/747; A61K 35/741; A61K 35/742

(52) U.S. Cl. CPC A61K 47/36; A61K 35/741; A61K 35/742; A61K 35/747; A61K 47/26

(57) ABSTRACT

Compositions and methods are disclosed for providing beneficial effects to animals, such as reducing harmful pathogens and increasing performance. In one embodiment, the composition is a gel that can be applied to a bird to increase performance and reduce pathogens. In yet another embodiment, the composition further comprises one or more direct fed microbial (“DFM”). In still another embodiment, methods are disclosed for treatment of and/or for the prevention of diseases of birds.

### CROSS-REFERENCE TO RELATED APPLICATIONS

This application is a continuation patent application of U.S. patent application Ser. No. 13/446,720 filed Apr. 13, 2012, which claims priority under 35 U.S.C. §119(e) to U.S. Provisional Patent Application No. 61/475,540 filed Apr. 14, 2011, the entirety of each application recited above is incorporated by reference herein.

### BIBLIOGRAPHY

Complete bibliographic citations of the references referred to herein by the first author's last name and year of publication in parentheses can be found in the Bibliography section, immediately preceding the claims.

### FIELD

The invention relates to compositions and methods for administering agents to birds.

### BACKGROUND

Several infections and diseases in poultry are caused by pathogenic bacteria, including E. coli, Clostridium, and Salmonella. Avian pathogenic E. coli (APEC) comprise a specific subset of pathogenic E. coli that cause extraintestinal diseases of poultry.

Clostridium affecting poultry include C. perfringens, C. septicum, and C. botulinum, which are anaerobic, gram-positive, spore-forming rods that produce potent toxins. Gangrenous dermatitis and cellulitis have reemerged recently as a significant concern for poultry producers in the U.S.

Thus, pathogenic bacteria are a major problem for poultry producers. Further complicating this situation is the fact that pathogen populations in poultry production facilities typically fluctuate in terms of both levels and types of pathogens, making control of the pathogens difficult. An adequate disease prevention program is essential to a profitable commercial poultry operation. Chronic diseases can reduce efficiency and increase costs

Agents can be administered to birds for a variety of reasons including preventing disease and stimulating growth. In some instances, this happens when chicks are a day old but it can also occur when birds are older.

Accordingly, there is a recognized need for alternatives, such as compositions and methods for treating or preventing disease in poultry. Furthermore, there is an important need for improving performance in and health of poultry.

### SUMMARY

Compositions and methods are disclosed for providing beneficial effects to animals, including but not limited to reducing harmful pathogens and increasing performance. In one embodiment, the composition is a gel that can be applied to a bird to increase performance and reduce pathogens. In yet another embodiment, the composition further comprises one or more direct fed microbial (“DFM”).

In one embodiment, the composition comprises one or more gum; one or more polysacharride; one or more monosacharride; and one or more dye. In another embodiment, the composition further comprises an adsorbent or absorbent. In still another embodiment, the composition comprises one or more DFM. In yet another embodiment, the DFM includes but is not limited to L. brevis strain 1E-1 ATCC Accession No. PTA-6509, B. subtilis strain LSSAO1 Accession No. NRRL B-50104, P. jensenii Accession No. NRRL B-30979 (P63), B. subtilis strain 15A-P4 Accession No. ATCC PTA-6507, and B. subtilis strain BS2084 Accession No. NRRL B-50013.

In one embodiment, the one or more gum comprises from about 6% to about 16% of the weight of the composition. In another embodiment, the one or more polysacharride comprises from about 50% to about 90% of the weight of the composition. In still another embodiment, the one or more monosacharride comprises from about 4% to about 10% of the weight of the composition. In yet another embodiment, the one or more dye comprises about 0.5% to about 3% of the weight of the composition.

In still another embodiment, a composition is disclosed comprising (percent of the weight of all gel ingredients, without including any agent or water) 4.7% xanthan gum, 4.7% guar gum, 7.9% dextrose, 79.3% corn starch, 1.3% dye, and 2% alkaline aluminosilicate. In another embodiment, the composition further comprises an effective amount of one or more DFMs. In one embodiment, the total amount of one or more DFMs is 5×10⁸ cfu/g. In another embodiment, the composition is applied to a bird and the total amount of one or more DFMs in the composition is 5×10⁸ cfu/bird.

In yet another embodiment, compositions disclosed have a viscosity of from about 250 cps to about 450 cps. In another embodiment, compositions disclosed have a viscosity of about 350 cps.

In still another embodiment, a method is disclosed comprising administering an effective amount of a composition to one or more bird. The composition comprises one or more gum; one or more polysacharride; one or more monosacharride; and one or more dye.

### BRIEF DESCRIPTION OF THE DRAWINGS

Exemplary embodiments of the invention are illustrated in the accompanying drawings, in which like reference numerals represent like parts throughout and in which:

FIG. 1A and FIG. 1B are an embodiment of a machine to apply a gel described herein onto birds.

FIGS. 2-4 are photographs of the machine of FIG. 1A and FIG. 1B.

FIG. 5 is a graph showing total lactic acid bacteria counts from gastrointestinal tracts of birds from Example 1.

FIG. 6 is a graph of poult weights (g) over time for the first fourteen days based on individual weights (treated n=75, control n=50) of birds from Example 1.

FIG. 7 is a graph of poult weights (g) on day fourteen based on individual weights (treated n=75, control n=50) of birds from Example 1. Treated weights were significantly higher (p=0.0329).

FIG. 8 is a graph of poult weights (g) on day forty-two based on average group weights (treated n=16, control n=16) of birds from Example 1. No significant difference was observed (p=0.0959).

FIG. 9 is a photograph of two petri dishes showing presumptive lactic growth in treated vs. control gut tracts (mucosa) of birds from Example 1.

Before explaining embodiments of the invention in detail, it is to be understood that the invention is not limited in its application to the details of construction and the arrangement of the components set forth in the following description or illustrated in the drawings. The invention is capable of other embodiments or being practiced or carried out in various ways. Also, it is to be understood that the phraseology and terminology employed herein is for the purpose of description and should not be regarded as limiting.

### DETAILED DESCRIPTION

The numerical ranges in this disclosure are approximate, and thus may include values outside of the range unless otherwise indicated. Numerical ranges include all values from and including the lower and the upper values, in increments of one unit, provided that there is a separation of at least two units between any lower value and any higher value. As an example, if a compositional, physical or other property, such as, for example, molecular weight, melt index, temperature etc., is from 100 to 1,000, it is intended that all individual values, such as 100, 101, 102, etc., and sub ranges, such as 100 to 144, 155 to 170, 197 to 200, etc., are expressly enumerated. For ranges containing values which are less than one or containing fractional numbers greater than one (e.g., 1.1, 1.5, etc.), one unit is considered to be 0.0001, 0.001, 0.01 or 0.1, as appropriate. For ranges containing single digit numbers less than ten (e.g., 1 to 5), one unit is typically considered to be 0.1. These are only examples of what is specifically intended, and all possible combinations of numerical values between the lowest value and the highest value enumerated, are to be considered to be expressly stated in this disclosure. Numerical ranges are provided within this disclosure for, among other things, relative amounts of components in a mixture, and various temperature and other parameter ranges recited in the methods.

The invention is directed to compositions and methods for delivering compositions to birds. In one embodiment, the composition is a high viscosity liquid. The liquid can be a soft flowable gel having a viscosity low enough such that the gel can be dispensed onto birds but high enough to form droplets when dispensed.

The gel can be used for administering agents. As used herein, the term “agent” means a chemical or biological entity that can be administered to an animal. The agent can have a property that induces a physiological response in an animal (e.g., humans, mice, rats, poultry, cows, horses, bird, and the like). The agent can comprise a single chemical or biological entity, combinations of chemical entities, combinations of biological entities, or combinations of chemical and biological entities. Non-limiting examples of agents include direct-fed microbials (DFMs), vaccines, competitive exclusion agents, antigens, peptides, immunomodulators, nutrients (e.g., fat, carbohydrate, protein, one or more bacterial strain, combinations of bacterial strains, vitamins, minerals), prebiotic compounds, botanicals, non-nutritive feed additives, and antibiotics.

“Effective amount,” as used herein with respect to the gel means a quantity of gel sufficient to dose at least about 90% of the birds to which the gel is applied.

“Effective amount,” as used herein with respect to the DFM(s) means a quantity of DFM(s) sufficient to improve performance (as defined herein) and/or to provide an anti-pathogenic effect.

In at least some embodiments, the gel includes one or more DFM(s). DFMs are bacteria that provide animals positive effects, including, but not limited to, reducing harmful pathogens and increasing performance. Performance measures include but are not limited to such parameters as average daily feed intake, average daily weight gain, total weight gain, European production factor, feed conversion, which includes both feed:gain and gain:feed, feed efficiency, mortality, and actual production costs.

In one embodiment, gel is applied to birds. The gel can be applied in any manner that produces the desired effect including but not limited to spraying, coating, painting, directed spot applications, spot treatments, and localized treatments.

In at least some embodiments, birds are chicks, and they are placed in a hatchling tray. In this embodiment, the gel is sprayed onto the birds while they are in the trays. Sprayed gel forms droplets that attach to the birds and the area surrounding them. Birds eat the droplets off themselves, other birds, and the area surrounding them, thereby consuming the gel and any agents added to the gel. Gel can be applied to birds of different ages. Gel can also be applied to poultry and exotic fowl, including, but not limited to, chicks, turkey poults, goslings, ducklings, guinea keets, pullets, hens, roosters (also known as cocks), cockerels, and capons.

In some embodiments, the gel includes one or more gum(s), such as xanthan gum, e.g., xanthan gum 40, xanthan gum 80, xanthan gum 200, and other xanthan gums, guar gum, e.g., guar gum 100, guar gum 175, guar gum 200, guar gum 225, guar gum 200/50, gum arabic, locust bean gum, and gum tragacanth. The gum contributes to the formation of the gel. In some embodiments, the gel includes a combination of xanthan gum, such as xanthan gum 80, and guar gum, such as guar gum 175.

One or more simple sugar(s), i.e., monosaccharide(s), such as dextrose, fructose, and galactose is/are included in some embodiments of the gel. In one embodiment, the gel includes dextrose (C₆H₁₂O₆), which is also known as D-glucose, glucose, or grape sugar. The simple sugar disperses gum(s) into water. It also improves solubility and eliminates product clumping of gel ingredients on water surface during ingredient rehydration. Where DFMs are used, the simple sugar(s) provide(s) considerably more consistent, as well as rapid distribution and suspension of the DFMs in the gel and therefore to the birds. DFM uniformity to the bird enhances proper dosing of the gel. Where DFMs and other microbes are used, the simple sugar(s) also provide(s) a readily utilizable microbial carbon source, which potentially enhances the viability of DFM cells.

At least some embodiments of the gel include one or more complex sugar(s), i.e., polysaccharide, such as cellulose, glycogen, and starch of any type, e.g., corn starch, wheat starch, and potato starch. In some embodiments, the gel includes corn starch. The complex sugar(s) retain(s) the size of the drop of gel once it is sprayed onto the birds and increase(s) the stickiness of the gel. The amount of complex sugar(s) that is/are used can be optimized to achieve the desired stickiness of the gel. The optimal level can be determined by eye. For instance, if the gel falls off the bird and/or if the gel leaves a trail on the bird instead of a droplet, the amount of complex sugar(s) can be increased.

One or more adsorbent(s) or absorbent(s) can also be added to the gel. Adsorbents and absorbents useful in the gel include, but are not limited to, bentonite, Fuller's earth, activated carbon, aerogels, and baylith. In some embodiments, Zeolite® baylith is used. Where one or more DFM(s) is used with the gel, the adsorbent(s) or absorbent(s) scavenge(s) water to increase the DFM's stability and viability during product storage.

The gel additionally can include one or more dye(s), such as green dye, blue dye, red dye, yellow dye, or any other dye. In at least some embodiments, the gel includes green and blue dyes, although the gel can be made with only one of these dyes or with other dyes. Dyes increase consumption of gel by the chicks because chicks are drawn to the dyed gel more than they are drawn to colorless gel. Dyes also can be used as a marker for analyzing consumption on a per bird basis. This can be accomplished by examining tongues of chicks to determine which birds ate the gel.

In one exemplary embodiment of the gel, the gel includes the following with amounts provided as percent of the weight of all gel ingredients (without including any agent or water): 6% to 16% of one or more gum(s) (total where more than one gum is used), 4% to 10% of one or more simple sugar(s) (total where more than one simple sugar is used), 50% to 90% of one or more complex sugar(s) (total where more than one complex sugar is used), 0.5% to 3% of one or more dye(s) (total where more than one dye is used), and 2% to 3% adsorbent(s) or absorbent(s) (or both) (total where more than one adsorbent or absorbent is used).

In another exemplary embodiment of the gel, the gel includes the following with amounts provided as percent of the weight of all gel ingredients (without including any agent or water): 8% to 12% of one or more gum(s) (total where more than one gum is used), 7% to 9% of one or more simple sugar(s) (total where more than one simple sugar is used), 70% to 87% of one or more complex sugar(s) (total where more than one complex sugar is used), 1% to 2% of one or more dye(s) (total where more than one dye is used), and 1% to 2% adsorbent(s) or absorbent(s) (or both) (total where more than one adsorbent or absorbent is used).

Another embodiment of the gel includes the following with amounts provided as percent of the weight of all gel ingredients (without including any agent or water): 5.1% xanthan gum 80 (Danisco USA, Inc., New Century, Kans.), 5.1% guar gum 175 (Danisco USA, Inc., New Century, Kans.), 8% dextrose (Archers Daniels Midland Company, Decatur, Ill.), 78.5% corn starch (NOVATION® 5600, National Starch Food Innovation, Bridgewater, N.J.), 1% dedusted green dye (for example, green shade dedusted from Sensient Technologies, Milwaukee, Wis.), 0.3% blue dye (for example, Blue No. 1, granular dm from Sensient Technologies, Milwaukee, Wis.), and 2% BAYLITH® zeolite (AB Colby, Inc., McMurray, Pa.).

In at least some embodiments, a viscosity of about 250 to about 450 centipoise (cps) is used. In one embodiment, a viscosity of about 350 cps is used. However, viscosities above and below these measurements can also be used. At about 250 to about 450 cps, gel ingredients mix into water without clumping.

In the embodiments of the gel in which both xanthan gum and guar gum are used, the ratio of xanthan gum to guar gum provides an appropriate viscosity level. In at least some embodiments, the ratio of xanthan gum to guar gum is 2:1 to 1:2. In some embodiments, the ratio of xanthan gum to guar gum is 1:1. These ratios of xanthan gum to guar gum require lower amounts of gums than using only one of the gums to achieve suitable viscosity of the gel. This provides a cost savings.

The gel has a viscosity that permits application of the gel to birds through application equipment. Two embodiments of application equipment are described below. However, other application equipment, such as the equipment described in U.S. Pat. No. 6,910,446, and in U.S. Published Patent Appln. No. 20080190373, the disclosures of both of which are incorporated herein by reference, can be used to apply the gel.

In another embodiment, methods of making the gel are disclosed. The gel is prepared by adding the gel ingredients to water or other suitable liquid. The gel ingredients include one or more than one of the following components: one or more gum; one or more polysacharride; one or more monosacharride; one or more dye; and one or more absorbent or adsorbent.

In another embodiment, one or more direct-fed microbial can mixed with the gel including but not limited to L. brevis strain 1E-1 ATCC Accession No. PTA-6509, B. subtilis strain LSSAO1 Accession No. NRRL B-50104, P. jensenii Accession No. NRRL B-30979 (P63), B. subtilis strain 15A-P4 Accession No. ATCC PTA-6507, and B. subtilis strain BS2084 Accession No. NRRL B-50013.

In some embodiments, from 50 g to 100 g of the combined gel ingredients is added to about 2.5 L of water. In other embodiments, from about 100 g to about 200 g of the combined gel ingredients is added to about 5.0 L of water. In one embodiment, about 77.0 g of the combined gel ingredients is added to about 2.5 L of water. In still another embodiment, about 191 g of the combined gel ingredients is added to about 5.0 L of water.

The gel ingredients are mixed into the water. An immersion blender can be used for this or any other suitable equipment.

Also provided herein are the following. A gel for use as a delivery carrier of active agents and/or biological agents characterized in that it comprises the composition according to the invention is also provided herein.

A gel for use in the treatment of and/or for the prevention of diseases of birds, including, but not limited to infectious bronchitis, infectious bursal disease, Marek's disease, necrotic enteritis, coccidiosis, and haemorrhagic enteritis, characterized in that it comprises the composition according to the invention is additionally provided herein.

A gel for use in the treatment of and/or for the prevention of diseases of birds, including, but not limited to, diseases and infections caused by bird pathogens, including, but not limited to, Clostridium perfringens, E. coli, and Salmonella sp. characterized in that it comprises the composition according to the invention is additionally provided herein.

A gel for use in the treatment of birds, including, but not limited to, improving performance (as defined herein) of the birds, characterized in that it comprises the composition according to the invention is additionally provided herein.

Also provided herein is a gel for use in the preparation of a medicament characterized in that it comprises the composition according to the invention.

Additionally provided herein is a use of a gel in the preparation of a delivery carrier of active agents and/or biological agents characterized in that it comprises the composition according to the invention.

The disclosure also provides a use of a gel in the preparation of a medicament characterized in that it comprises the composition according to the invention.

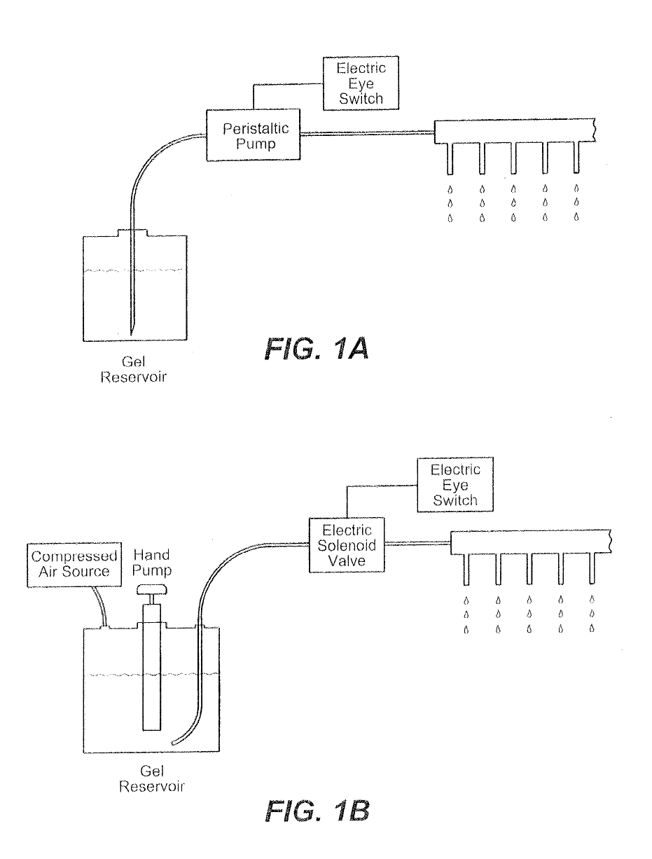

Referring now to FIGS. 1-4, an exemplary machine for administering a gel will now be described. The terms machine and gel applicator are used interchangeably herein. In at least one embodiment of the machine, the machine generates droplets rather than spray or beadlets. The droplets are in the form of a curtain of droplets. The embodiment includes delivery through a tubular bar or cylinder having a set of evenly-spaced delivery tubes. The tubes are fluidically coupled to a gel reservoir. The small evenly-spaced delivery tubes deliver an accurate amount of small droplets of gel directly onto birds. In at least some embodiments, the volume of each bead is about 50 μl.

In some hatcheries, day-old chicks are processed using conveyors that move them from one station to the next. For example, chicks are moved from a sexing station to a vaccination station using a conveyor. When this machine is used in such a setting, application of the droplets can occur during the final conveyor and stacking process or at any other step in the processing of day-old chicks. In this embodiment of the machine, volumetric doses of the gel are controlled by pneumatic pressure over hydraulics in a low pressure container. This can be achieved in conjunction with an electric solenoid valve and an electric eye switch, as shown in FIG. 1.

In another embodiment of the machine, volumetric dosing is achieved by a non-pressurized system of a variable speed peristaltic pump. The pump can include an electric eye switch, as shown in FIG. 1.

Liquid flow is controlled by an electric solenoid valve actuated by a normally open photo cell switch triggered by beam interruption, such as during tray processing before stacking. Conveyor speed, height of gel droplet fall, pressure, and orifice size and number ensure dosage, accuracy, and placement of droplets onto every bird, which typically means gel droplets fall on 96+% of the birds.

A second embodiment of the machine is used for delivering lower dosages. This embodiment employs a variable ratio peristaltic pump with a double head. The same application bar is used as with the first embodiment of the machine, but smaller orifice nozzlettes are used in the second embodiment.

In at least some embodiments of using the gel, the gel is applied at a rate of about 20 ml to about 35 ml of gel per 100 birds using a suitable machine, such as the one described above. In at least some embodiments, about 28 ml of the gel is applied per 100 birds using a suitable machine, such as the one described above.

Where DFMs are used with the gel, the target application of the DFM is about 1×10⁵ to about 1×10¹² cfu/bird, with the cfu being the colony forming units of the DFM. The cfu is the total cfu where more than one DFM is administered. In at least some embodiments of using the gel, the target application of the DFM is more than or equal to about 5.0×10⁸ cfu/bird. When applied to birds, the birds have a high rate of consumption of gel. This consumption rate has been observed to be over 90%.

In some embodiments, one more of the following DFM(s) is added to the gel: Lactobacillus brevis strain 1E-1, Propionibacteria jensenii strain P63, Bacillus strains 3A-P4, 15A-P4, 22C-P1, BS27, 2084, and LSSA01.

Lactobacillus brevis strain 1E-1 was deposited under the Budapest Treaty on Jan. 12, 2005 at the American Type Culture Collection, 10801 University Blvd., Manassas, Va. 20110, under accession number PTA-6509.

Propionibacteria jensenii strain P63 was deposited on Jan. 15, 2009, in the Deutsche Sammlung von Mikroorganismen and Zellkulturen GmbH (DSMZ, Inhoffenstr. 7 B, D-38124 Braunschweig, Germany) under number DSM22192 by Danisco Deutschland GmbH (Bush-Johannsen-Str. 1, 25899 Niebüll, Germany).

On Jan. 12, 2005, strains 3A-P4, 15A-P4, and 22C-P1 were deposited at the American Type Culture Collection (ATCC), 10801 University Blvd., Manassas, Va. 20110-2209 and given accession numbers PTA-6506 (3A-P4), PTA-6507 (15A-P4), and PTA-6508 (22C-P1), respectively. Strains 2084, LSSAO1, and BS 27 were deposited on Mar. 8, 2007, Jan. 22, 2008, and Jan. 24, 2008, respectively, at the Agricultural Research Service Culture Collection (NRRL), 1815 North University Street, Peoria, Ill., 61604 and given accession numbers NRRL B-50013, NRRL B-50104, and NRRL B-50105, respectively. All deposits were done under the Budapest Treaty.

In other embodiments, one or more other DFM can be added to the gel. For example, DFMs that have previously been described are suitable for use in the invention. Such DFMs have been described, for example, in U.S. Pat. Nos. 7,618,640; 7,384,628; 5,945,333; 6,951,643; 6,221,650; 7,354,757; 7,754,469; 8,021,654; 8,025,874; 6,455,063; U.S. Patent Publication Nos. 2010/017,2873 and 2010/018,3574; and U.S. Ser. Nos. 12/498,734 and 13/110,529.

In another embodiment, the gel includes the following with amounts provided as percent of the weight of all gel ingredients (without including any agent or water): 4.7% xanthan gum 80 (Danisco USA, Inc., New Century, Kans.), 4.7% guar gum 175 (Danisco USA, Inc., New Century, Kans.), 7.9% dextrose (Archers Daniels Midland Company, Decatur, Ill.), 79.3% corn starch (NOVATION® 5600, National Starch Food Innovation, Bridgewater, N.J.), 1% food grade coloring green dye (for example, “Green Shade Dedusted,” from Sensient Technologies, Milwaukee, Wis.), 0.3% food grade coloring blue dye (for example, Blue No. 1, granular dm from Sensient Technologies, Milwaukee, Wis.), 2% alkaline aluminosilicate, and an effective amount of one or more DFM. In one embodiment, the total amount of DFM is 5×10⁸ cfu/bird.

In another embodiment, the DFMs are L. brevis, ATCC PTA-6509 and P. jensenii, NRRL B-30979 (P63). In still another embodiment, the gel comprises L. brevis totaling 2.5×10⁸ cfu/bird and P63 totaling 2.5×10⁸ cfu/bird.

In another embodiment, the DFMs are 1E1 (L. brevis, ATCC PTA-6509) and LSSA01 (B. subtilis). In still another embodiment, the gel comprises 500,000,000 (5.0×10⁸) cfu/bird of 1E1 (L. brevis, ATCC PTA-6509, 2.5×10⁸ cfu/bird) and LSSA01 (B. subtilis, NRRL B-50104, 2.5×10⁸ cfu/bird).

In yet another embodiment, the DFMs in the gel are 15A-P4 (B. subtilis, ATCC PTA-6507), LSSA01 (B. subtilis, NRRL B-50104) and BS2084 (B. subtilis, NRRL B-50013) strain combination. In still another embodiment, DFMs in the gel are 15A-P4 (B. subtilis, ATCC PTA-6507, 1.67×10⁸ cfu/bird), LSSA01 (B. subtilis, NRRL B-50104, 1.67×10⁸ cfu/bird) and BS2084 (B. subtilis, NRRL B-50013, 1.67×10⁸ cfu/bird) strain combination, respectively.

In one embodiment, gel comprising at least one DFM can be used to improve growth performance and feed intake of birds.

In another embodiment, gel comprising at least one DFM with at least one Propionibacteria can be used to increase mucosal attached beneficial Propionibacteria.

In another embodiment, gel comprising at least one DFM can be used to decrease pathogens associated with a bird. In still another embodiment, gel comprising at least one DFM can be used to decrease mucosal attached pathogens.

In still another embodiment, gel comprising at least one DFM can be used to decrease mucosal attached C. perfringens and avian pathogenic E. coli.

### EXAMPLES

The following Examples are provided for illustrative purposes only. The Examples are included herein solely to aid in a more complete understanding of the presently described invention. The Examples do not limit the scope of the invention described or claimed herein in any fashion.

### Example 1

### Brief Summary

Lactobacillus and Propionibacteria levels in the gut mucosa were significantly higher (p<0.05) in treated group vs. control group on days 0 and 1, becoming equalized by day 7 (p=0.13). When analyzed as means of the individual poults weights, treated poults were heavier (p=0.033) at day 14. No other significant weight differences were observed between treated and control birds on days 0, 1, or 7. No Clostridium spp. bacteria were detected on day 0, 1, 7, or 14 samples. No significant differences were observed between total E. coli counts between the treated vs. control groups on any day of sampling. Mortalities were lower in control poult houses at both days 7, 14 and the cumulative days 0-14 totals.

### Objectives

Determine the effect of DFMs Lactobacillus brevis strain 1E-1 and Propionibacteria jensenii strain P63 on weight, mortality and in reducing poults E. coli challenges post-hatch (Day 1, 1 hr after treatment) day 2, day 7, day 14 and day 42 benchmarks. DFMs delivered in a gel (described below).

### Materials and Methods

Details for this Trial:

All birds were from the same hatchery. The coccidial control was Coyden (Huvepharma, Sofia, Bulgaria). Birds received penicillin via water at day 7-11. No DFMs were used in the controls. No coccidial vaccines were used. No feed antibiotics were used. Therapeutics were used at discretion of a veterinarian. Other deviations, changes, and observations throughout the trial were recorded.

### General Procedures:

Treatments are listed below:

| | | |
|---|---|---|
| 1 | TREATED | Treated with Lactobacillus brevis strain 1E1 and |
| | | Propionibacterium jensenii P63 delivered in gel |
| 2 | CONTROL | Untreated control (No DFM or gel) |

DFM strains were administered to poults in trays of 100 in the hatchery at a dose of 10,000 birds/bottle (2.5 L of water) to achieve an approximate dosage of 5×10⁸ CFU total DFM per poult. The DFM was 14% by weight Lactobacillus brevis strain 1E-1 and 36% by weight Propionibacteria jensenii strain P63, with 50 g of the combined DFMs used. 1.30×10¹¹ CFU of Lactobacillus brevis strain 1E-1 was used. 1.30×10¹¹ CFU of Propionibacteria jensenii strain P63 was used. The other 50% is composed of gel ingredients as follows.

| Gel Ingredients | % |
|---|---|
| Xanthum 80 | 5 |
| Guar 175 | 5 |
| Yeast Extract | 2 |
| Dextrose | 5 |
| Starch (Novation | 30 |
| 5600-Corn) | |
| Green Dye (MX 135) | 2 |
| Blue Dye | 1 |
| Baylith | 1 |
| Carrier Total | 50 |

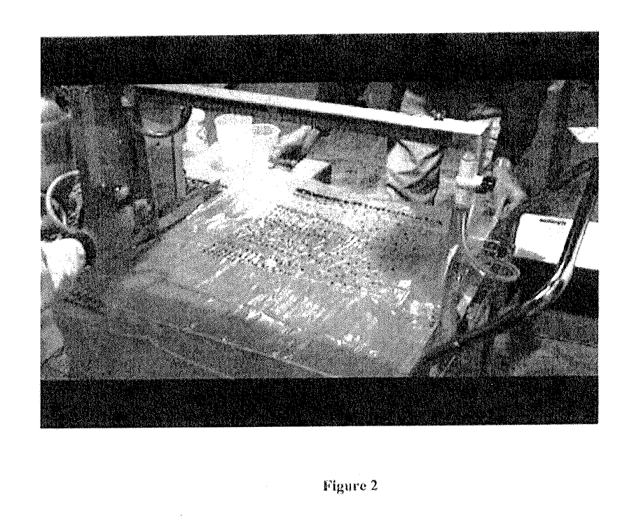

The total weight on the gel ingredients was 50 g. The gel ingredients and the DFMs were placed in a bag. Each bag of gel ingredients (including the DFMs) was dissolved in 2.5 liters of water. DFMs in the gel were administered via of a metered pump delivery system, as shown in FIGS. 2-4.

### Flock Info

Flocks 604L1 (5900) and 606L1 (1100) in each group Nicholas Line 85 Toms weighed at hatchery, day 1 on farm, day 7, and day 14 (all weights in grams).

### Microbial Analyses of GIT:

The following were analyzed: duodenum, jejunum of the mid-gut, approximately 3 inches on both sides of the Meckel's diverticulum, and the ileum with NO rinsing, but cut longitudinally to expose contents in 10 birds per treatment for Total E. coli, APEC, Clostridia, Lactobacillus, and Propionibacteria at Day 1 one hour after treatment (10 pre-treatment birds and 10 treated), Day 2, Day 7, and Day 14. In addition, the liver and the yolk sac were analyzed separately. Samples were processed and saved for community analysis if needed. Samples were collected in sterile Whirl-Pak filter bags liver and yolk sac were collected in separate bags.

### Procedure:

Birds were placed and treated on day 0, one hour after treatment. Ten birds per treatment were shipped to the lab (20 birds total) for analysis. Same amount of birds were shipped for analysis on day 1 of placement, day 7 and day 14. Mortality and body weight will also be recorded on those sampling days and afterward.

### Dispensing the Gel

Gel was dispensed using gel applicator at a rate of 28 ml/100 birds. FIGS. 2-4 show the experiment described in this example and in particular, the machine used to apply gel to chicks. FIG. 2 shows a machine used to apply gel onto birds, with the gel being applied to a substrate. FIG. 3 shows the machine applying gel onto hatchling trays. FIG. 4 shows the machine applying gel to chicks in hatchling trays. The machine is described in the text above the Examples.

Summary (Samples Include Liver, Yolk Sac, and GITs without Ceca):

| | Actions | Details |
|---|---|---|
| Day 0 | Apply gel to treated | Samples taken 1 hr after application. |
| | (and not to control) | Treated, Control (10 each), weight and |
| | today of hatch poults. | mortality |
| Day1 | Sample 1 day after | Samples taken: Treated, Control (10 |
| | hatch (day of | each) weight and mortality |
| | placement). | |
| Day 7 | Sample 7 days after | Samples taken: Treated, Control (10 |
| | hatch. | each) weight and mortality |
| Day 14 | Sample 14 days | Samples taken: Treated, Control (10 |
| | after hatch. | each) weight and mortality |

### Microbial Analytical Procedures

Entire GITs minus ceca, yolks sac and livers for Day 0, Day 1, Day 7 and Day 14, were dissected, and placed in sterile whirl-Pak filter bags, then shipped to the laboratory for analyses within 24 hours of collection. Once at the laboratory, the duodenum, jejunum (of the mid-gut, approximately 3 inches on both sides of the Meckel's diverticulum), and the ileum were cut longitudinally to expose the contents, placed back into the sterile Whirl-Pak bag and weighed. All samples were masticated for 60 seconds with 99 ml sterile peptone water. The samples were then plated at dilutions 10⁻¹ and 10⁻³ on CHROM agar and Perfringens agar base for the enumeration of E. coli and Clostridium, respectively. On Day 14 the mucosa and the digesta were used.

### Statistical Analysis

Bacterial counts were transformed to the log₁₀ value and analyzed using a one way Analysis of Variance (ANOVA) using GraphPad Prism 5 software and comparing all pairs of columns with parametric methods where significance is defined as P value <0.05. Samples for microbial analysis below the limit of detection were assigned a value of 1.0 cfu (log₁₀=0).

### Results

### Microbial Analysis

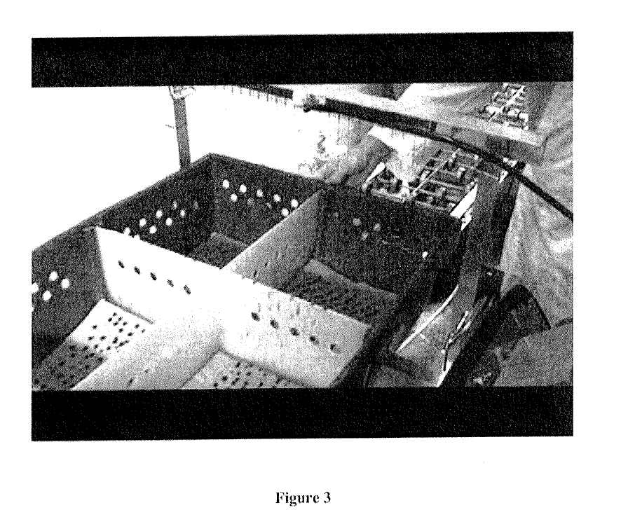

Higher levels of total lactic acid bacteria and propionibacteria were observed in GITs from treated samples at days 0 and 1 compared to the untreated control poults. (See Table 1 below and FIG. 5).

TABLE 1
Lactobacillus, Propionibacteria and total E. coli level in gastrointestinal
tract mucosal sections. From each group n = 10 samples.

| | log₁₀ CFU/g mucosa | | | | | |
|---|---|---|---|---|---|---|
| | Total Lactic | | Total | | | |
| | Acid Bacteria | | Propionibacteria | | Total E. coli | |
| | Treated | control | Treated | control | Treated | control |
| Day 0 | 6.42^{a} | 2.83^{b} | 5.71 | 2.10 | 0.00 | 0.00 |
| Day 1 | 6.76^{a} | 4.63^{b} | 5.46 | 3.77 | 0.00 | 0.00 |
| Day 7 | 7.30^{a} | 6.70^{a} | 0.00 | 0.00 | 5.37 | 5.05 |
| Day 14* | 8.57^{a} | 8.13^{a} | 0.00 | 0.00 | 4.16 | 4.58 |

In Table 1, where superscripts are the same, e.g., ^{a}and^{a}, the results are not statistically significant, and where superscripts are different, e.g., ^{a}and^{b}, the results are statistically significant.

Lactobacillus and Propionibacteria were found in the yolk sac of treated and control on day 1 only; (log₁₀ 7.99/7.54 and log₁₀ 3.43/2.79 for (1) lactics and propionibacteria treated and (2) control, respectively) but levels found in the yolk sac were not statistically different (p>0.05). No other yolk samples on days 0 had detectable levels. Yolk sacs by days 7 and 14 had been reabsorbed. E. coli was not detected in any yolk sample tested.

None of the organisms assayed were found in the liver over any time point or treatment. No Clostridium spp. were detected in any sample over time and treatment.

### Weight and Mortality Comparisons

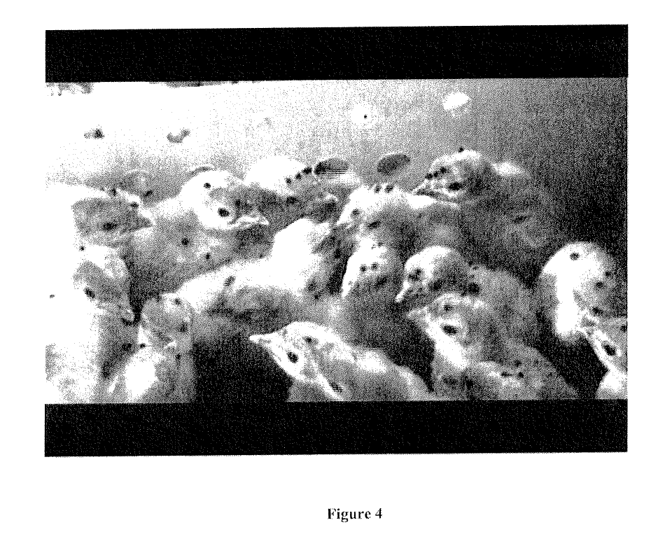

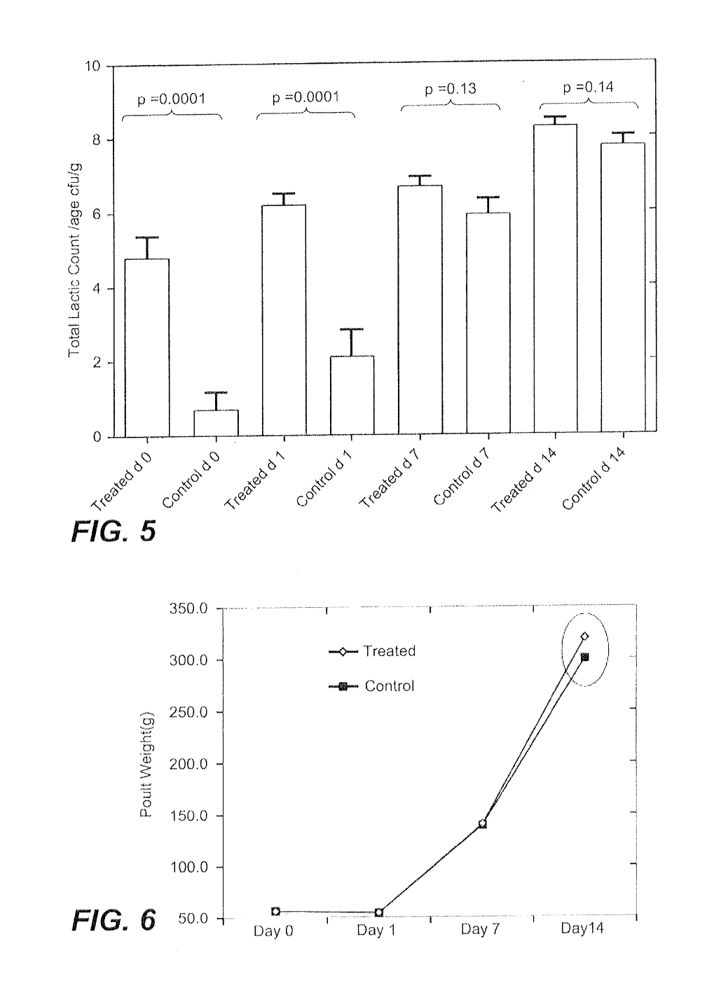

Compared to control, a significant body weight gained of 6.3% was observed at day 14 based on individual birds' weight. Based on individual weights, average weight for treated birds was 319.50 g compared to 300.20 g for control birds (p<0.05), an increase of 6.3%. Table 2 below shows average poult weight (g) and mortality by sampling day treated vs. control groups. Statistical comparison of individual poult weights is indicated in pairings with either the same or different superscripts indicating significance (p<0.05). FIG. 6 is a graph showing poult weights (g) over time for the first fourteen days based on individual weights (treated n=75, control n=50) of birds. FIG. 7 is a graph of poult weights (g) on day fourteen based on individual weights (treated n=75, control n=50) of birds. Treated weights were significantly higher (p=0.0329). Calculated as combined average weights, day 14 groups also showed an increased weight gain in the treated group (314.1 g) vs. control group (306.6 g).

Mortalities observed in treated vs. control house were as follows: days 0-7 treated vs. control: 2.41% vs. 1.66%, days 7-14 treated vs. control: 0.66% vs. 0.63% and cumulative days 0 to 14 treated vs. control: 3.16% vs. 2.30%.

Percentage livability observed at day forty two was as follows: treated vs. control: 96.13% vs. 96.06%

TABLE 2

| | In- | | | | | |
|---|---|---|---|---|---|---|
| | dividual | Individual | Group | Group | Percentage | Percentage |
| | Weights | Weights | weights | weights | Mortality | Mortality |
| | Treated | Control | Treated | Control | Treated | Control |
| Day 0 | 54.61^{a} | 56.44^{a} | n.d | n.d | n.d. | n.d. |
| Day 1 | 54.29^{a} | 53.50^{a} | 57.04 | 57.21 | n.d. | n.d. |
| Day 7 | 137.55^{a} | 139.96^{a} | 134.47 | 136.68 | 2.41 | 1.66 |
| Day 14 | 319.50^{a} | 300.20^{b} | 304.13 | 311.42 | 0.66 | 0.63 |

In Table 2, where superscripts are the same, e.g., ^{a}and^{a}, the results are not statistically significant, and where superscripts are different, e.g., ^{a}and^{b}, the results are statistically significant.

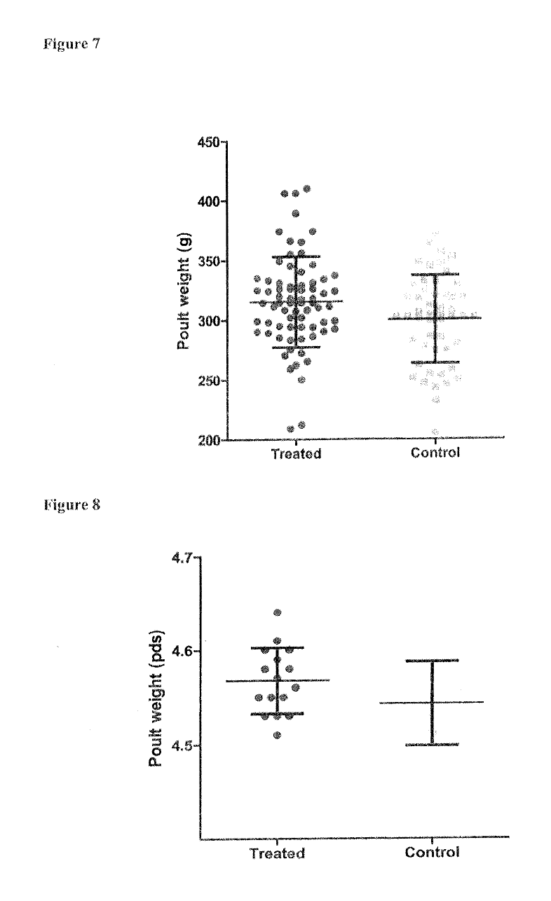

When analyzed as means of the group poults weights on day 42, no significant difference was observed between both group (p=0.959) at day 14. FIG. 8 is a graph of poult weights (g) on day 42 based on average group weights (treated n=16, control n=16) of birds. Birds were weighed in groups of five birds (instead of individually) on day 42. No significant difference was observed (p=0.0959)). However, treatment tended (p<0.10) to increase bird performance compared to control.

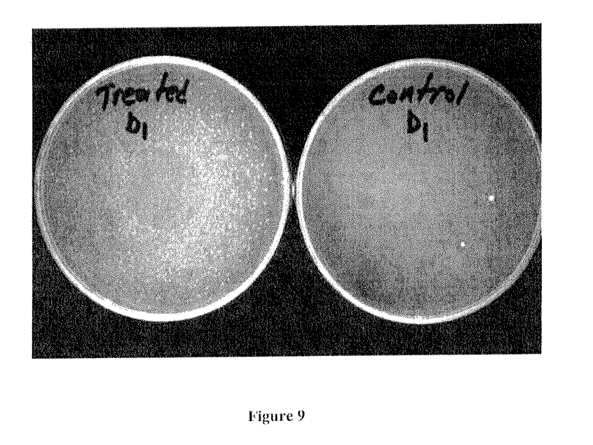

FIG. 9 shows presumptive lactic growth in treated vs. control gut tracts (mucosa) on MRS agar plates. MRS agar is a medium that selects for total lactic acid bacteria. The plates have gut tract homogenate from (1) poults treated on Day 0 with DFMs Lactobacillus brevis strain 1E-1 and Propionibacteria jensenii strain P63 and (2) control poults. Both were harvested on Day 1.

### Conclusions

The consumption of the Lactobacillus brevis strain 1E1 and Propionibacterium jensenii P63, which was applied in the gel, resulted in a higher count of Lactobacillus and Propionibacteria in the GITs for the treated birds compared to control.

While not evident until day 14, a weight advantage was observed between the treated and control poults.

Both treated and control houses required antibiotic therapy post day 14.

### Example 2

### Objectives

This experiment will test a gel having a different formulation than the gel in Example 1.

### Materials and Methods

### Gel

The composition and proportion of the ingredients in the gel will be as recited in Table 3.

TABLE 3
Composition and proportion of ingredients in the gel.

| Carrier Ingredients | % | Supplier | Manufacturer |
|---|---|---|---|
| Xanthan 80 | 5.1 | Danisco | Danisco |
| Guar 175 | 5.1 | Danisco | Danisco |
| Dextrose | 8 | Food Ingredients | ADM |
| Starch (Novation 5600-Corn) | 78.5 | | |
| Green Shade Dedusted | 1 | Sensient | Sensient |
| Blue No. 1 Granular DM | 0.3 | Sensient | Sensient |
| Baylith | 2 | AB Colby | AB Colby |
| Carrier Total | 100 | | |

Information about the ingredient suppliers is provided above.

About 77 g of the gel ingredients will be added to 2.5 L of water. The gel ingredients and one or more DFM will be mixed into the water with an immersion blender or any suitable instrument.

### DFMs

One or more DFM will be included in the gel. The total amount of DFM (whether it is one DFM or more than one) will be added so that 1×10⁵ CFU to 1×10¹² CFU per bird is delivered.

### Dispensing the Gel

The gel will be applied at a rate of 20 ml to 35 ml of gel per 100 birds using a suitable machine, such as one described above.

The gel will be applied to day-old chicks. Chicks will be positioned in a hatchling tray, box, or other suitable device for holding chicks. The tray containing the chicks will be placed on a conveyor belt, and the chicks will be propelled toward the gel applicator. Gel will be delivered directly onto the chicks as they travel past pressurized nozzles, nozzlettes, or orifices in the applicator. The chicks will consume the gel and any DFM or other agent contained therein.

### Expected Results

It is expected that gel described in this example will have increased stickiness when compared to the gel of Example 1. This will improve consumption of the gel and therefore any DFM or other agent included in the gel by the chicks. It is expected that tongue checks for dye will show about 95% with green stain. It is also expected that chicks will consume most gel droplets within about 2-3 minutes of application.

It is understood that the various preferred embodiments are shown and described above to illustrate different possible features of the invention and the varying ways in which these features may be combined. Apart from combining the different features of the above embodiments in varying ways, other modifications are also considered to be within the scope of the invention. The invention is not intended to be limited to the preferred embodiments described above.

### Example 3

### Objectives

This experiment determined the efficacy and potential benefits of a High Viscosity Gel with DFMs.

### Materials and Methods

One day after hatch, 80 broiler chickens were derived from local commercial hatchery and transported to University of Wisconsin Madison Poultry Science test facility. At test facility, chicks were split into two treatment groups. The control group contained forty chicks that were placed in 5 comfort battery cages containing 8 chicks each.

The treated group contained chicks on HVL with 1E1 & P63 that were treated one time with 30 mL/bird High Viscosity Liquid (HVL, 190.75 g with 4.7% xanthan gum, 4.7% guar gum, 7.9% Dextrose, 79.3% starch, 1.0% food grade coloring “Green Shade Dedusted”, 0.3% Food grade coloring “Blue No. 1 Granular DM” and 2% alkaline aluminosilicate in 5 L of water), containing a total of 500,000,000 (5.0×10⁸) cfu of strains 1E1 (L. brevis, ATCC PTA-6509, totals 2.5×10⁸ cfu/g) and P63 (P. jensenii, NRRL B-30979, totals 2.5×10⁸ cfu/g).

Gelatinous HVL was applied via gel drip applicator. After treatment, 40 treated chicks were assigned to 5 replicate comfort cages containing 8 birds per pen. All birds were fed standard commercial feed throughout the trial. Two birds per replicate were sacrificed on day 1 for microbial baseline determination and confirmation of treatment. Performance was determined via average daily gain (ADG, in grams), average daily feed intake (ADFI, in grams) and feed conversion rate (Feed to Gain, F/G or Gain to Feed, G/F) on days 7 and 14. On day 14, birds were sacrificed to permit collection of digestive tract mucosa for determination of microbial load of bacterial groups of interest via traditional microbial enumeration procedures. Statistical analysis was performed using SAS 9.1.3 Proc Mixed procedure. Data is presented distinguishing significance level α1=0.05 indicated by differing superscripts, and α2=0.10 indicated by differing superscripts in brackets. The trial protocol was approved by University of Wisconsin Animal Care Committee.

### Results

Significant performance differences (P<0.05) in absolute body weight were determined on day 14, indicating that birds treated with HVL and strain P63 and 1E1 combination performed better than control birds (Table 4). This improvement of performance (P<0.05) was also observed in ADG of 2^{nd} trial week (Table 5) and cumulative performance data (Table 6), which was accompanied with significant increase (P<0.05) of feed intake compared with control animals. Performance within first week of trial showed numerical differences only (P>0.10; Table 7). Feed efficiency was not impacted (P>0.10) by treatment.

TABLE 4
Average body weight by treatment.¹

| Treatment | N | Day 1 | Day 7 | Day 14 |
|---|---|---|---|---|
| Control | 5 | 51.4 | 161.7 | 358.7^{a} |
| HVL with 1E1 & | 5 | 53.2 | 167.9 | 471.2^{b} |
| P63 | | | | |
| P-Value | | >.100 | >.100 | 0.029 |
| SEM | | 1.1 | 4.6 | 27.4 |

¹in grams; SEM, standard error of the mean; superscripts indicate difference at significance level α = 0.05.

TABLE 5
Performance parameters of second week of trial.

| Treatment | N | ADG¹ | ADFI¹ | F:G¹ | G:F¹ |
|---|---|---|---|---|---|
| Control | 5 | 28.2^{a} | 28.2^{a} | 1.610 | 0.667 |
| HVL with | 5 | 43.3^{b} | 43.3^{b} | 1.293 | 0.774 |
| 1E1 & P63 | | | | | |
| P-Value | | 0.021 | 0.016 | >.100 | >.100 |
| SEM | | 3.6 | 3.4 | 0.131 | 0.039 |

¹ADG, average daily gain in grams; ADFI, average daily feed intake in grams; F:G, feed to gain; G:F, gain to feed; SEM, standard error of the mean; superscripts in brackets indicate difference at significance level α = 0.10, superscripts indicate difference at significance α = 0.05

TABLE 6
Performance parameters of complete duration of the trial.

| Treatment | N | ADG | ADFI | F:G | G:F |
|---|---|---|---|---|---|
| Control | 5 | 22.0^{a} | 32.7^{a} | 1.549 | 0.662 |
| HVL with | 5 | 29.9^{b} | 41.2^{b} | 1.383 | 0.723 |
| 1E1 & P63 | | | | | |
| P-Value | | 0.032 | 0.015 | >.100 | >.100 |
| SEM | | 1.9 | 1.9 | 0.069 | 0.025 |

Superscripts indicate difference at significance level α = 0.05.

TABLE 7
Performance parameters of first week of trial.

| Treatment | N | ADG | ADFI | F:G | G:F |
|---|---|---|---|---|---|
| Control | 5 | 15.8 | 24.2 | 1.539 | 0.651 |
| HVL with 1E1 | 5 | 16.4 | 26.4 | 1.618 | 0.619 |
| & P63 | | | | | |
| P-Value | | >.100 | >.100 | >.100 | >.100 |
| SEM | | 0.6 | 0.9 | 0.032 | 0.012 |

Analysis of trial birds for bacterial composition of harmful and beneficial bacteria groups on day 14 revealed significant higher (P < .001) Propionibacterium counts in HVL with 1E1 & P63 treatment only (Table 8). Avian pathogenic E. coli and C. perfringens were numerically reduced (P > 0.10) compared with control birds.

TABLE 8
Harmful and beneficial bacteria load on day 14.¹

| Treatment | N | APEC | CP | LAB | Prop |
|---|---|---|---|---|---|
| Control | 5 | 3.45 | 0.61 | 6.45 | 0.30^{a} |
| HVL with 1E1 & | 5 | 2.88 | 0.00 | 6.61 | 4.30^{b} |
| P63 | | | | | |
| P-Value | | >.100 | >.100 | >.100 | <.001 |
| SEM | | 0.30 | 0.31 | 0.11 | 0.49 |

¹Data in log₁₀ cfu; APEC, Avian pathogenic E. coli (pathogenic), CP, C. perfringens (pathogenic), LAB, lactic acid bacteria (beneficial), Prop, Propionibacterium (beneficial); superscripts indicate difference at significance level α = 0.05; SEM, standard error of the mean.

### Conclusions

The experiments above demonstrate that a one time application of HVL gel with 1E1 & P63 on day 1 after hatch improves growth performance and feed intake of birds. In addition, a one time application of HVL gel with 1E1 & P63 on day 1 after hatch also increased mucosal attached beneficial Propionibacteria. Finally, HVL gel with 1E1 & P63 on day 1 after hatch numerically decreased mucosal attached C. perfringens and avian pathogenic E. coli.

### Example 4

### Objectives

This experiment determined the benefits of a High Viscosity Liquid with DFMs and compared the effects to several other treatment groups.

### Materials and Methods

One day after hatch, one hundred and twenty (120) broiler chickens were derived from local commercial hatchery and transported to University of Wisconsin Madison Poultry Science test facility. At test facility, chicks were split into three treatment groups. In the first treatment group, forty chicks were treated one time with 30 mL/bird High Viscosity Liquid (HVL, 190.75 g with 4.7% xanthan gum, 4.7% guar gum, 7.9% Dextrose, 79.3% starch, 1.0% food grade coloring “Green Shade Dedusted”, 0.3% Food grade coloring “Blue No. 1 Granular DM” and 2% alkaline aluminosilicate in 5 L of water) without direct fed microbial. The HVL was applied via gel drip applicator to the chick and then the chicks were placed in 5 comfort battery cages (8 chicks per cage).

In the second treatment group, forty chicks were treated with HVL with DFMs. The chicks in this treatment group received similar amounts of HVL via gel drip applicator as for first group but the treatment also contained a total of 500,000,000 (5.0×10⁸) cfu of either 1E1 (L. brevis, ATCC PTA-6509, 2.5×10⁸ cfu) and LSSA01 (B. subtilis, NRRL B-50104, 2.5×10⁸ cfu) strain combination or 15A-P4 (B. subtilis, ATCC PTA-6507, 1.67×10⁸ cfu), LSSA01 (B. subtilis, NRRL B-50104, 1.67×10⁸ cfu) and BS2084 (B. subtilis, NRRL B-50013, 1.67×10⁸) strain combination, respectively.

In the third treatment group, forty chicks were treated with similar amounts of HVL via gel drip applicator as the first group but contained EnvivaPro™ from Danisco Animal Nutrition.

After treatment, 8 chicks per replicate were assigned to 5 replicate pens each and fed standard commercial broiler feed. Two birds per replicate were sacrificed on day 1 for microbial baseline determination and confirmation of treatment. Performance was determined via average daily gain (ADG, in grams), average daily feed intake (ADFI, in grams) and feed conversion rate (Feed to Gain, F/G or Gain to Feed, G/F) on days 7 and 14.

In addition to growth performance, 2 birds per replicate and time point were sacrificed to permit collection of digestive tract mucosa for determination of microbial load of bacterial groups of interest via traditional microbial enumeration procedures. Statistical analysis was performed using SAS 9.1.3 Proc Mixed procedure. Data is presented distinguishing significance level α1=0.05 indicated by differing superscripts, and α2=0.10 indicated by differing superscripts in brackets. Means were separated by least square difference (LSD) procedure. The trial protocol was approved by University of Wisconsin Animal Care Committee.

### Results

Significant differences (P<0.10) were observed in total body weight at days 7 and 14 (Table 9). Average daily gain and ADFI were significantly increased (P<0.05) due to treatment for complete duration of trial (Table 10), but showed differences in patterns over time.

TABLE 9
Average body weight by treatment.¹

| Treatment | N | day 1 | day 7 | day 14 |
|---|---|---|---|---|
| Control (HVL only) | 5 | 48.8 | 155.4^{(a)} | 378.0^{a} |
| HVL with 1E1 & A01 | 5 | 50.9 | 173.2^{(b)} | 450.6^{b} |
| HVL with Enviva Pro | 5 | 50.2 | 169.0^{(ab)} | 483.5^{b} |
| P-Value | | >.100 | 0.076 | 0.018 |
| SEM | | 0.6 | 24.2 | 118.4 |

¹in grams; SEM, standard error of the mean; superscripts indicate difference at significance level α1 = 0.05 and α2 = 0.10 in brackets; separation of means by LSD.

TABLE 10
Performance parameters of complete duration of the trial.¹

| Treatment | n | ADG | ADFI | F:G | G:F |
|---|---|---|---|---|---|
| Control (HVL | 5 | 23.5^{a} | 35.5^{a} | 1.520 | 0.659 |
| only) | | | | | |
| HVL with 1E1 | 5 | 28.5^{b} | 42.5^{b} | 1.485 | 0.675 |
| & A01 | | | | | |
| HVL with | 5 | 30.9^{b} | 43.8^{b} | 1.422 | 0.704 |
| Enviva Pro | | | | | |
| P-Value | | 0.020 | 0.039 | >.100 | >.100 |
| SEM | | 8.4 | 10.6 | 0.144 | 0.065 |

¹ADG, average daily gain in grams; ADFI, average daily feed intake in grams; F:G, feed to gain; G:F, gain to feed; SEM, standard error of the mean; superscripts indicate difference at significance level α1 = 0.05; separation of means by LSD.

Lactobacillus and Bacillus containing treatment HVL with 1E1 & A01 showed its major impact in the first week of the trial (Table 11), whereas solely Bacillus based treatment HVL with EnvivaPro demonstrated its impact on performance and feed intake in the second week of the trial (Table 12). Feed efficiency was not impacted (P>0.10) by treatment in this study.

TABLE 11
Performance parameters of first week of trial.¹

| Treatment | N | ADG | ADFI | F:G | G:F |
|---|---|---|---|---|---|
| Control (HVL | 5 | 15.2^{(a)} | 23.0^{a} | 1.517 | 0.660 |
| only) | | | | | |
| HVL with 1E1 | 5 | 17.5^{(b)} | 27.3^{b} | 1.568 | 0.639 |
| & A01 | | | | | |
| HVL with | 5 | 17.0^{(ab)} | 26.2^{ab} | 1.547 | 0.647 |
| Enviva Pro | | | | | |
| P-Value | | 0.099 | 0.020 | >.100 | >.100 |
| SEM | | 3.2 | 4.9 | 0.104 | 0.043 |

¹ADG, average daily gain in grams; ADFI, average daily feed intake in grams; F:G, feed to gain; G:F, gain to feed; SEM, standard error of the mean; superscripts indicate difference at significance level α1 = 0.05 and α2 = 0.10 in brackets; separation of means by LSD.

TABLE 12
Performance parameters of second week of trial.¹

| Treatment | n | ADG | ADFI | F:G | G:F |
|---|---|---|---|---|---|
| Control (HVL | 5 | 31.8^{a} | 47.9^{(a)} | 1.542 | 0.654 |
| only) | | | | | |
| HVL with 1E1 | 5 | 39.6^{ab} | 57.6^{(ab)} | 1.449 | 0.692 |
| & A01 | | | | | |
| HVL with | 5 | 44.9^{b} | 61.5^{(b)} | 1.379 | 0.728 |
| Enviva Pro | | | | | |
| P-Value | | 0.034 | 0.078 | >.100 | >.100 |
| SEM | | 15.4 | 18.2 | 0.243 | 0.105 |

¹ADG, average daily gain in grams; ADFI, average daily feed intake in grams; F:G, feed to gain; G:F, gain to feed; SEM, standard error of the mean; superscripts indicate difference at significance level α1 = 0.05 and α2 = 0.10 in brackets; separation of means by LSD.

There were no significant differences (P>0.10) observed due to treatment over the duration of the trail, but clear numerical differences towards reduction of Avian Pathogenic E. coli (APEC) were shown (Table 13). Clostridium perfringens was not determined in trial animals.

TABLE 13
Harmful and beneficial bacteria load on days 7 and 14.¹

| | | Day 7 | | | | Day 14 | | | |
|---|---|---|---|---|---|---|---|---|---|
| Treatment | n | APEC | CP | LAB | Prop | APEC | CP | LAB | Prop |
| Control (HVL | 5 | 6.410 | b.d. | 8.413 | 0.538 | 6.218 | b.d. | 7.715 | 1.884 |
| only) | | | | | | | | | |
| HVL with 1E1 & | 5 | 6.072 | b.d. | 8.415 | 0.536 | 5.830 | b.d. | 7.733 | 1.014 |
| A01 | | | | | | | | | |
| HVL with | 5 | 5.924 | b.d. | 8.398 | 0.391 | 5.799 | b.d. | 7.535 | 0.891 |
| EnvivaPro | | | | | | | | | |
| P-Value | | >.100 | — | >.100 | >.100 | >.100 | — | >.100 | >.100 |
| SEM | | 0.116 | — | 0.101 | 0.275 | 0.139 | — | 0.170 | 0.394 |

¹Data in log₁₀ cfu; APEC, Avian pathogenic E. coli (pathogenic), CP, C. perfringens (pathogenic), LAB, lactic acid bacteria (beneficial), Prop, Propionibacterium (beneficial); b.d., below detection of 1 × 10³ cfu in all individual samples; SEM, standard error of the mean.

### Conclusions

The data above show that a one time application of HVL gel with strains 1E1 & A01 improves growth performance of birds and reduces mucosal attachment of APEC.

In addition, one time application of HVL gel with EnvivaPro improves growth performance of birds and numerically reduces mucosal attached APEC.

### Example 5

### Objectives

This experiment determined the benefits of a High Viscosity Liquid with DFMs in a conventional production system.

### Materials and Methods

Broiler chickens were hatched at commercial hatchery according to standard commercial operating procedure. Treatment was applied for three consecutive weeks in a conventional production system using antibiotic growth promoters (AGP). Treatments consisted of the following: (1) no HVL application (Control); (2) in ovum Gentamicin injection (Gentamicin); (3) HVL application with strains L. brevis 1E1 and Bacillus subtilis LSSA01 direct fed microbials (see Example 4 for detail); and (4) a combination of in ovum Gentamicin injection and HVL with strains L. brevis 1E1 and Bacillus subtilis LSSA01 direct fed microbials (Gent&HVL+DFM).

Treatments were each applied to 12 flocks per week. Each flock consisted of more than 45,000 head each. Performance was tracked per flock for standard commercial performance at slaughter around 5 week of production corrected by average performance of production site in order to account for site differences. Measures determined were average daily gain (ADG in grams), average daily feed intake (ADFI in grams), feed conversion rate (F:G, feed to gain ratio; G:F, gain to feed ratio), European production factor (EPF [(ADG×% survival rate)/Feed Conversion×10], day 3 and 7 mortality (in %) and actual production costs relative to control treatment (in %).

For flocks being sampled for bacterial load information, bacterial load baseline was determined on day 1 of production in order to normalize mucosa bacterial load data according to bacterial load at start of trial due to differences of pathogen status in breeder flocks. Mucosa bacterial load of representative beneficial and harmful bacteria groups was compared on days 7 and 14.

### Results

Statistical analysis was performed using SAS 9.1.3 Proc Mixed procedure. Data is presented distinguishing significance level α1=0.05 indicated by differing superscripts, and α2=0.10 indicated by differing superscripts in brackets. Means were separated by Tukey honest significant difference (HSD) procedure.

Over the complete duration of the trial, there was a numerical improvement (P>0.10) observed in ADG in HVL with DFM treatment (Table 14), which also showed highest (P<0.05) ADFI compared with combined Gentamicin and HVL DFM treatment. European production factor, accounting for a number of factors relevant to production, was numerically higher (P>0.10) in treatments containing Gentamicin (Table 14).

Combined Gentamicin and HVL DFM treatment showed the highest (P<0.10) feed conversion rate as well as numerically lowest (P>0.10) day 3 and 7 mortality compared with control treatment (Table 15). Actual production costs were lowest (P<0.05) in Gentamicin and DFM treated flocks (Table 15).

TABLE 14
Standard production performance measures in twelve flocks per
treatment with an approximate 45,000 head per flock.¹

| | | ADG | ADFI | | | |
|---|---|---|---|---|---|---|
| Treatment | N | (g) | (g) | F/G | G/F | EPF |
| Control | 12 | 57.8 | 107.1^{ab} | 1.852^{(b)} | 0.540^{(a)} | 296 |
| Gentamicin | 12 | 58.0 | 106.1^{ab} | 1.828^{(ab)} | 0.547^{(ab)} | 304 |
| HVL with DFM | 12 | 59.0 | 108.9^{b} | 1.846^{(ab)} | 0.542^{(ab)} | 295 |
| Gent&HVL + | 12 | 57.6 | 104.6^{a} | 1.816^{(a)} | 0.551^{(b)} | 303 |
| DFM | | | | | | |
| P-Value | | >.100 | 0.028 | 0.095 | 0.095 | >.100 |
| SEM | | 0.2 | 0.5 | 0.006 | 0.002 | 2 |

¹ADG: average daily gain in grams; ADFI: average daily feed intake in grams; F:G: feed to gain; G:F: gain to feed; EPF: European production factor; superscripts indicate difference at significance level α1 = 0.05, superscripts in brackets indicate difference at significance level α2 = 0.10; separation of means by Tukey HSD; Gent& HVL + DFM, in ovum Gentamicin application followed by direct fed microbial in high viscosity liquid application post hatch; SEM, standard error of the mean.

TABLE 15
Measurements of mortality and actual production costs

| | | | Day 7 | |
|---|---|---|---|---|
| | | Day 3 | mort¹ | |
| Treatment | N | mort¹ (%) | (%) | % Cost |
| Control | 12 | 0.50 | 1.07 | 100.0^{b} |
| Gentamicin | 12 | 0.43 | 0.90 | 97.9^{ab} |
| HVL with DFM | 12 | 0.45 | 0.92 | 100.7^{b} |
| Gent&HVL + DFM | 12 | 0.40 | 0.89 | 95.0^{a} |
| P-Value | | >.100 | >.100 | 0.032 |
| SEM | | 0.04 | 0.07 | 0.8 |

¹mort, mortality; superscripts indicate difference at significance level α1 = 0.05, superscripts in brackets indicate difference at significance level α2 = 0.10, separation of means by Tukey HSD

Since previous research indicated differences in commensal microbiota composition due to treatment, avian pathogenic E. coli (APEC) and C. perfringens (CP) as well as beneficial lactic acid bacteria (LAB) and Propionibacteria (Props) were enumerated at day 7 (Table 16) and at day 14 (Table 17). HVL DFM treatment alone showed lowest (P<0.05) pathogen load on both sampling days (Tables 16 and 17). On day 7, APEC was highest (P<0.05) in Gentamicin treated birds, but lactic acid bacteria (LAB, beneficial) was increased (P<0.001) in Gentamicin only treatment.

TABLE 16
Mucosa bacterial load of pathogenic and beneficial bacteria on day 7.¹

| | | day 7 | | | |
|---|---|---|---|---|---|
| Treatment | N | APEC | CP | LAB | Prop |
| Control | 6 | 3.59^{ab} | 0.45^{b} | 3.58^{a} | 1.99^{b} |
| Gentamicin | 6 | 5.12^{b} | 0.21^{ab} | 5.06^{b} | 0.38^{a} |
| HVL with DFM | 6 | 1.08^{a} | 0.06^{a} | 3.59^{a} | 1.17^{ab} |
| HVL + DFM&Gent | 6 | 4.69^{b} | 0.46^{b} | 3.73^{a} | 0.99^{ab} |
| P-Value | | 0.022 | 0.032 | <.001 | 0.027 |
| SEM | | 0.54 | 0.04 | 0.16 | 0.20 |

¹Data in log₁₀ cfu; separation of means by Tukey HSD; superscripts indicate difference at significance level α1 = 0.05; superscripts in brackets indicate difference at significance level α2 = 0.10; APEC: Avian pathogenic E. coli (pathogenic); CP: C. perfringens (pathogenic); LAB: lactic acid bacteria (beneficial); Prop: Propionibacterium (beneficial); SEM, standard error of the mean.

TABLE 17
Mucosa bacterial load of pathogenic and beneficial bacteria on day 14.¹

| | | day 14 | | | |
|---|---|---|---|---|---|
| Treatment | N | APEC | CP | LAB | Prop |
| Control | 6 | 1.67^{ab} | 0.66^{ab} | 3.59 | 0.73^{ab} |
| Gentamicin | 6 | 4.25^{bc} | 0.41^{a} | 4.47 | 0.29^{a} |
| HVL with DFM | 6 | 1.22^{a} | 0.25^{a} | 4.15 | 0.87^{b} |
| HVL + DFM&Gent | 6 | 5.09^{c} | 0.94^{b} | 3.58 | 0.99^{b} |
| P-Value | | 0.002 | 0.013 | >.100 | 0.009 |
| SEM | | 0.48 | 0.09 | 0.20 | 0.08 |

¹Data in log₁₀ cfu; separation of means by Tukey HSD; superscripts indicate difference at significance level α1 = 0.05; superscripts in brackets indicate difference at significance level α2 = 0.10; APEC: Avian pathogenic E. coli (pathogenic); CP: C. perfringens (pathogenic); LAB: lactic acid bacteria (beneficial); Prop: Propionibacterium (beneficial); SEM, standard error of the mean.

HVL DFM treatment alone was lowest (P<0.001) in LAB counts, which was in part due to the proven superiority of L. brevis 1E1 (Gebert, et al.). Since Lactobacillus in general have been shown to counteract E. coli (Barefoot et al., Berg et al., Cintas et al., Fang et al., Hugo et al., Jin et al. (2000), Jin et al. (1996), Lewus et al., Reid et al. and Vold et al.), the low day 7 counts (P<0.001) of LAB in most efficient performing animals (P<0.10; Table 16) from combined Gentamicin and HVL DFM treatment group prove an increase in LAB quality rather than a quantitative LAB difference.

Propionibacteria were lowest (P<0.05) due to Gentamicin treatment on both sampling days. Once again, HVL DFM was able to improve negative effects of Gentamicin by increasing Prop counts on day 7 and actively promoting Props on day 14 (P<0.01) to highest levels compared with negative impact of Gentamicin treatment alone.

### Conclusions

The above data demonstrate that a one time application on day 1 after hatch with an HVL gel with strains 1E1 and A01 reduces APEC and CP pathogen load in gut mucosa of week old chicks in conventional production. Further, the one time application of HVL gel with 1E1 & A01 on day 1 after hatch increases beneficial, slow growing Props in gut mucosa of chicks around 14 days in production with AGP.

In addition, the HVL gel with DFMs can be used in conjunction with other treatments, such as an antibiotic. Application of the HVL gel with DFM strains 1E1 and A01 in combination with in ovum gentamicin application improves feed efficiency and reduces production costs after 5 weeks of production. Similarly, one time application of HVL gel with strains 1E1 & A01 in combination with in ovum gentamicin application is able to maintain bird performance in spite of high pathogenic load in gut mucosa caused by gentamicin during 5 week production cycle.

### Example 6

### Objectives

This experiment determined the effects and benefits of a High Viscosity Liquid with DFMs in an antibiotic free production system.

### Materials and Methods

Broiler chickens were hatched at commercial hatchery according to standard commercial operating procedure. Treatment was applied for three consecutive weeks in an antibiotic free production system. Treatments consisted of no HVL application (Control), treatment with HVL only, and HVL application with strains L. brevis 1E1 and Bacillus subtilis LSSA01 direct fed microbials (for detail on HVL with DFM, see Example 4).

Treatments were each applied to 8 flocks per week. Each flock consisted of more than 45,000 head each. Performance was tracked per flock for standard commercial performance at slaughter around 5 week of production, and was corrected by average performance of production site in order to account for flock differences. Measures determined were average daily gain (ADG in grams), average daily feed intake (ADFI in grams), feed conversion rate (F:G, feed to gain ratio; G:F, gain to feed ratio), European production factor (EPF [(ADG×% survival rate)/Feed Conversion×10], day 3 and 7 mortality (in %) and actual production costs relative to control treatment (in %). For flocks being sampled for bacterial load information, bacterial load baseline was determined on day 1 of production in order to normalize mucosa bacterial load data according to bacterial load at start of trial due to differences of pathogen status in breeder flocks. Mucosa bacterial load of representative beneficial and harmful bacteria groups was compared on days 7 and 14.

Statistical analysis was performed using SAS 9.1.3 Proc Mixed procedure. Data is presented distinguishing significance level α1=0.05 indicated by differing superscripts, and α2=0.10 indicated by differing superscripts in brackets. Means were separated by least square difference (LSD) procedure.

### Results

Five week performance of ABF birds on trial is shown in Table 18. Mortality and production costs are shown in Table 19. Application of HVL only to broiler chickens showed reduction (P<0.05) in feed efficiency and highest (P<0.001) production costs. Applying HVL with DFM to ABF broiler chickens showed improved (P<0.05) growth performance and highest (P<0.001) feed intake compared with other treatment groups at intermediate feed efficiency (see Tables 18 and 19).

European production factor and overall mortality was improved (P<0.10) in HVL DFM treatment compared with other treatment groups. Actual production costs were lowest (P<001) in HVL with DFM birds compared with control and HVL only treatments.

TABLE 18
Week 5 performance of 8 flocks per treatment with approximately 45,000
birds per flock in antibiotic free production system.¹

| | | ADG | ADFI | | | |
|---|---|---|---|---|---|---|
| Treatment | N | (g) | (g) | F/G | G/F | EPF |
| No HVL | 8 | 58.6^{a} | 105.4^{a} | 1.800^{a} | 0.556^{a} | 307^{(a)} |
| HVL only | 8 | 58.5^{a} | 106.5^{b} | 1.821^{b} | 0.549^{b} | 305^{(a)} |
| HVL with DFM | 8 | 59.7^{b} | 108.3^{c} | 1.814^{ab} | 0.551^{ab} | 314^{(b)} |
| P-Value | | 0.012 | <.001 | 0.039 | 0.039 | 0.091 |
| SEM | | 0.196 | 0.317 | 0.00 | 0.001 | 2 |

¹ADG: average daily gain in grams; ADFI: average daily feed intake in grams; F:G, feed to gain; G:F, gain to feed; EPF: European production factor; superscripts indicate difference at significance level α1 = 0.05; superscripts in brackets indicate difference at significance level α2 = 0.10; separation of means by LSD; SEM, standard error of the mean.

TABLE 19
Mortality and actual production costs in antibiotic free production system.

| | | Day 3 | Day 7 | |
|---|---|---|---|---|
| Treatment | N | mort (%) | mort (%) | % Cost |
| No HVL | 8 | 0.80^{b} | 1.51^{b} | 100.0^{b} |
| HVL only | 8 | 0.71^{b} | 1.50^{b} | 102.3^{c} |
| HVL with DFM | 8 | 0.37^{a} | 0.76^{a} | 97.4^{a} |
| P-Value | | <.001 | <.001 | <.001 |
| SEM | | 0.05 | 0.09 | 0.5 |

mort, mortality;
superscripts indicate difference at significance level α1 = 0.05,
superscripts in brackets indicate difference at significance level α2 = 0.10;
separation of means by LSD;
SEM, standard error of the mean.

The effects of treatment were reflected well in day 7 bacterial load in bird mucosa (Table 20). Whereas HVL application alone lead to highest (P<0.05) APEC and CP counts, APEC and CP counts were lowest in HVL DFM treatment. HVL application alone reduced (P<0.06) LAB counts on days 7 and 14. Propionibacteria were numerically highest (P>0.10) as determined before in conventional production.

TABLE 20
Harmful and beneficial mucosal bacteria load on days 7 and 14.¹

| | | day 7 | | | | day 14 | | | |
|---|---|---|---|---|---|---|---|---|---|
| Treatment | N | APEC | CP | LAB | Prop | APEC | CP | LAB | Prop |
| No HVL | 6 | 4.72^{b} | 0.66^{ab} | 4.01^{b} | 0.63 | 2.72 | 0.96 | 3.85^{(b)} | 0.59 |
| HVL only | 6 | 5.78^{b} | 1.07^{b} | 3.56^{a} | 0.33 | 2.55 | 0.81 | 3.28^{(a)} | 0.48 |
| HVL with DFM | 6 | 2.26^{a} | 0.50^{a} | 3.93^{b} | 1.06 | 3.26 | 0.64 | 3.82^{(b)} | 0.70 |
| P-Value | | 0.026 | 0.054 | 0.013 | >.100 | >.100 | >.100 | 0.055 | >.100 |
| SEM | | 0.49 | 0.09 | 0.05 | 0.17 | 0.41 | 0.11 | 0.09 | 0.06 |

¹Data in log₁₀ cfu, separation of means by LSD, superscripts indicate difference at significance level α1 = 0.05, superscripts in brackets indicate difference at significance level α2 = 0.10; APEC, Avian pathogenic E. coli (pathogenic), CP, C. perfringens (pathogenic), LAB, lactic acid bacteria (beneficial), Prop, Propionibacterium (beneficial); SEM, standard error of the mean.

### Conclusions

The above data demonstrate that a one time application of HVL gel with strains 1E1 and A01 on day 1 after hatch reduces APEC and CP pathogen load in gut mucosa of week old chicks in antibiotic free production. Further, a one time application of HVL gel with strains 1E1 and A01 on day 1 after hatch increases overall animal performance in antibiotic free production.

The data further show that a one time application of HVL gel with strains 1E1 and A01 on day 1 after hatch decreases day 3 and 7 mortality in antibiotic free production. Finally, a one time application of HVL gel with strains 1E1 and A01 on day 1 after hatch reduces production costs after 5 weeks of production.

Although specific embodiments have been illustrated and described herein, it will be appreciated by those of ordinary skill in the art that any arrangement that is calculated to achieve the same purpose may be substituted for the specific embodiments shown. This application is intended to cover any adaptations or variations that operate according to the principles of the invention as described. Therefore, it is intended that this invention be limited only by the claims and the equivalents thereof. The disclosures of patents, references and publications cited in the application are incorporated by reference in their entirety herein.

### BIBLIOGRAPHY

1. Barefoot, S. F., and T. R. Klaenhammer. 1983. Detection and activity of lactacin B, a bacteriocin produced by Lactobacillus acidophilus. Applied and Environmental Microbiology 45:1808-1815.
2. Berg, R. D., and W. E. Owens. 1979. Inhibition of translocation of viable Escherichia coli from the gastrointestinal tract of mice by bacterial antagonism. Infection and Immunity 25:820-827.
3. Cintas, L. M., M. P. Casaus, C. Herranz, I. F. Nes, and P. E. HernÃ [symbol] ndez. 2001. Review: Bacteriocins of Lactic Acid Bacteria. Food Science and Technology International 7:281-305.
4. Fang, W., M. Shi, L. Huang, J. Chen, and Y. Wang. 1996. Antagonism of lactic acid bacteria towards Staphylococcus aureus and Escherichia coli on agar plates and in milk. Vet Res 27:3-12.
5. Gebert, S., E. Davis, T. Rehberger, and C. Maxwell. Lactobacillus brevis strain 1E1 administered to piglets through milk supplementation prior to weaning maintains intestinal integrity after the weaning event. Beneficial Microbes 2:35.
6. Hugo, A. A., E. Kakisu, G. L. De Antoni, and P. F. Perez. 2008. Lactobacilli antagonize biological effects of enterohaemorrhagic Escherichia coli in vitro. Letters in Applied Microbiology 46:613.
7. Jin, L.-Z., R. R. Marquardt, and S. K. Baidoo. 2000. Inhibition of enterotoxigenic Escherichia coli K88, K99 and 987P by the Lactobacillus isolates from porcine intestine. Journal of the Science of Food and Agriculture 80:619.
8. Jin, L. Z., Y. W. Ho, N. Abdullah, M. A. Ali, and S. Jalaludin. 1996. Antagonistic effects of intestinal Lactobacillus isolates on pathogens of chicken. Letters in Applied Microbiology 23:67.
9. Lewus, C. B., A. Kaiser, and T. J. Montville. 1991. Inhibition of food-borne bacterial pathogens by bacteriocins from lactic acid bacteria isolated from meat. Applied and Environmental Microbiology 57:1683-1688.
10. Reid, G., and J. Burton. 2002. Use of Lactobacillus to prevent infection by pathogenic bacteria. Microbes and Infection 4:319.
11. Vold, L., A. Hoick, Y. Wasteson, and H. Nissen. 2000. High levels of background flora inhibits growth of Escherichia coli O157:H7 in ground beef. International Journal of Food Microbiology 56:219.

What is claimed is:

1. A composition comprising:
one or more gum;
one or more polysacharride;
one or more monosacharride; and
one or more dye.

2. The composition of claim 1 further comprising one or more direct-fed microbial selected from the group consisting of: L. brevis strain 1E-1 ATCC Accession No. PTA-6509, B. subtilis strain LSSAO1 Accession No. NRRL B-50104, P. jensenii Accession No. NRRL B-30979 (P63), B. subtilis strain 15A-P4 Accession No. ATCC PTA-6507, and B. subtilis strain BS2084 Accession No. NRRL B-50013.

3. The composition of claim 1 further comprising direct-fed microbials L. brevis 1E-1 ATCC Accession No. PTA-6509 and P. jensenii Accession No. NRRL B-30979 (P63).

4. The composition of claim 1 further comprising direct-fed microbials L. brevis 1E-1 ATCC Accession No. PTA-6509 and B. subtilis strain LSSAO1 Accession No. NRRL B-50104.

5. The composition of claim 1 further comprising direct-fed microbials B. subtilis strain 15A-P4 Accession No. ATCC PTA-6507, B. subtilis strain LSSAO1 Accession No. NRRL B-50104, and B. subtilis strain BS2084 Accession No. NRRL B-50013.

6. The composition of claim 1, wherein the one or more gum comprises xanthan gum and guar gum.

7. The composition of claim 1, wherein the one or more polysacharride comprises starch.

8. The composition of claim 7, wherein the starch comprises cornstarch.

9. The composition of claim 1, wherein the one or more monosacharride comprises dextrose.

10. The composition of claim 1, wherein the one or more dye comprises a green dye and a blue dye.

11. The composition of claim 1, wherein the one or more gum comprises from about 6% to about 16% of the weight of the composition.

12. The composition of claim 1, wherein the one or more polysacharride comprises from about 50% to about 90% of the weight of the composition.

13. The composition of claim 1, wherein the one or more monosacharride comprises from about 4% to about 10% of the weight of the composition.

14. The composition of claim 1, wherein the one or more dye comprises about 0.5% to about 3% of the weight of the composition.

15. The composition of claim 1, wherein:
the one or more gum comprises from about 8% to about 12% of the weight of the composition;
the one or more polysacharride comprises from about 70% to about 87% of the weight of the composition;
the one or more monosacharride comprises from about 7% to about 9% of the weight of the composition; and
the one or more dye comprises from about 1% to about 2% of the weight of the composition.

16. The composition of claim 1, further comprising one or more adsorbent or absorbent, wherein:
the one or more gum comprises from about 9.4% of the weight of the composition;
the one or more polysacharride comprises from about 79.3% of the weight of the composition;
the one or more monosacharride comprises from about 7.9% of the weight of the composition;
the one or more dye comprises from about 1.3% of the weight of the composition; and
the one or more adsorbent or absorbent comprises about 2% of the weight of the composition.

17. The composition of claim 16, further comprising water, wherein the water is included at a ratio of about 100 g to about 200 g of the non-water composition ingredients to about 5.0 liters (about 5,000 grams) of water.

18. The composition of claim 16, wherein the composition has a viscosity of about 350 cps.

19. A method comprising administering a composition to one or more bird, wherein the composition comprises one or more gum; one or more polysacharride; one or more monosacharride; and one or more dye.

20. The method of claim 19, wherein the composition further comprises one or more direct-fed microbial selected from the group consisting of L. brevis strain 1E-1 ATCC Accession No. PTA-6509, B. subtilis strain LSSAO1 Accession No. NRRL B-50104, P. jensenii Accession No. NRRL B-30979 (P63), B. subtilis strain 15A-P4 Accession No. ATCC PTA-6507, and B. subtilis strain BS2084 Accession No. NRRL B-50013.
